SEQUENCE LISTING (3 sequences)

SEQ ID NO: 1 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 22 bp, Artificial
  PCR primer
taatcagacaaggaactgatta

SEQ ID NO: 3 - DNA, 19 bp, Artificial
  PCR primer
cgaaggtgtgacttccatg

(19) United States
(12) Patent Application Publication
(10) Pub. No.: US 2023/0364033 A1
(43) Pub. Date: Nov. 16, 2023

(54) COMPOUNDS FOR TREATING VIRUS INFECTIONS

(71) Applicant: Meletios Therapeutics, Paris (FR)

(72) Inventors: Xavier MANIERE, Montrouge (FR); Marc SALOME, Mervilla (FR)

(21) Appl. No.: 18/026,461

(22) PCT Filed: Sep. 17, 2021
(86) PCT No.: PCT/EP2021/075679

§ 371 Date: Mar. 15, 2023

(30) Foreign Application Priority Data
Sep. 17, 2020 (EP) 20306048.8
Feb. 18, 2021 (EP) 21305200.4

(51) Int. Cl. A61K 31/135; A61K 45/06; A61P 31/14

(52) U.S. Cl. CPC A61K 31/135; A61K 45/06; A61P 31/14

(57) ABSTRACT

The present invention relates to a compound of the following formula (I):

or a pharmaceutically acceptable salt, ester, hydrate, derivative, prodrug or metabolite thereof for use in the prevention or treatment of an infection by a virus in an individual.

### DOMAIN OF THE INVENTION

The present invention relates to compounds and methods for treating infections by viruses, in particular belonging to the Coronaviridae family, more particularly by the virus SARS-CoV-2.

### TECHNICAL BACKGROUND

In December 2019 an outbreak of pneumonia cases of unknown origin occurred in Wuhan in China and spread quickly nationwide. On Jan. 7, 2020, the causative pathogen was identified as a novel coronavirus, which was named 2019-nCoV and later SARS-CoV-2.

The new virus is closely related to both SARS-CoV (82% nucleotide identity) and MERS-CoV (50% nucleotide identity), yet distinct from them.

Early mortality rates suggested that COVID-19, the name for the disease caused by SARS-CoV-2, may be less severe than SARS and MERS. However, illness onset among rapidly increasing numbers of people rapidly suggested that SARS-CoV-2 would be more contagious than both SARS-CoV and MERS-CoV. As of May 11, 2020, 4 063 525 cases of COVID-19 (in accordance with the applied case definitions and testing strategies in the affected countries) have been reported, including 282 244 deaths.

A great deal of effort has been made to find effective drugs against the virus. Among the various compounds tested, remdesivir, a drug previously developed for the treatment of Ebola virus infections, has been reported to show promising efficacy and acceptable safety in treating COVID-19 in a news release of the National Institutes of Health (NIH) dated Apr. 29, 2020. As such, preliminary results indicate that patients who received remdesivir had a 31% faster time to recovery than those who received placebo (p<0.001). Specifically, the median time to recovery was 11 days for patients treated with remdesivir compared with 15 days for those who received placebo. Results also suggested a survival benefit, with a mortality rate of 8.0% for the group receiving remdesivir versus 11.6% for the placebo group (p=0.059).

However, the efficacy of remdesivir is not completely established yet as Wang et al. (2020) Lancet doi.org/10.1016/50140-6736(20)31022-9 report that in their trial remdesivir use was not associated with a difference in time to clinical improvement.

Early in the COVID-19 pandemic, hydroxychloroquine, a Sigma-1 receptor ligand, was proposed as a treatment of SARS-CoV-2 infections. However, its therapeutic activity is controversial (Kaptein et al. (2020) Proc. Natl. Acad. Sci. 117:26955-26965).

Accordingly, there is still a need for alternative treatments of infections by SARS-CoV-2.

### SUMMARY OF THE INVENTION

The present invention arises from the unexpected finding, by the inventors, that SR-31747 could be effective for treating infection by viruses, in particular by SARS-CoV-2.

Accordingly, the present invention relates to a compound of the following formula (I):

wherein
R1 represents a hydrogen atom or a halogen atom;
R2 represents a cyclohexyl or a phenyl;
R3 represents a cycloalkyl containing from 3 to 6 carbon atoms;
R4 represents a hydrogen atom, an alkyl containing from 1 to 6 carbon atoms or a cycloalkyl containing from 3 to 6 carbon atoms;
A represents a group selected from —CO—CH₂—, —CH(CI)—CH₂—, —CH(OH)—CH₂—, —CH₂—CH₂—, —CH═CH—, —C═C—;
   in particular SR-31747,
   or a pharmaceutically acceptable salt, ester, hydrate, derivative, prodrug or metabolite thereof for use in the prevention or treatment of an infection by a virus, in particular of the Coronaviridae family, in an individual.

The present invention also relates to a compound of the following formula (II):

wherein
Aa is un group selected from —CO—CH₂—, —CH(OH)—CH₂, —CH═CH—, —C═C—;
R1a represents a hydrogen atom or a halogen atom;
R2a is a cyclohexyl;
   or a pharmaceutically acceptable salt, ester, hydrate, derivative, prodrug or metabolite thereof, for use in the prevention or treatment of an infection by a virus, in particular of the Coronaviridae family, in an individual.

The present invention also relates to a compound of the following formula (III):

wherein
R1 b represents a hydrogen atom or a halogen atom;
R2b is a cyclohexyl group;
R3b represents a hydrogen atom or an alkyl group having 1 to 3 carbon atoms,
R4b represents an alkyl group having 1 to 3 carbon atoms which may the same or different than the alkyl group of R3b,
R3b and R4b considered together can form with the nitrogen atom to which they are attached, a heterocyclic group having from 5 to 7 atoms in the cycle, selected from piperidino, morpholino and pyrrolidino;
Ab represents a group —CH₂—CH₂— or —CH═CH—;
   or a pharmaceutically acceptable salt, ester, hydrate, derivative, prodrug or metabolite thereof, for use in the prevention or treatment of an infection by a virus, in particular of the Coronaviridae family, in an individual.

The present invention also relates to a compound of the following formula (IV):

wherein
Ar represents a phenyl, a naphthyl, a substituted phenyl or a substituted naphthyl group;
n is an integer included inclusively between 1 and 4;
RB represents an alkyl group provided that Ac represents a single bond and RA and RC, which may the same or different, independently represent a hydrogen atom or a group selected from a halogen atom, an alkyl, an alkyl substituted by one or more halogen atoms, and an alkoxy group;
or RB and RC together form a —(CH₂)p- bridge with p representing 0, 1 or 2 provided that RA represents a hydroxy or alkoxy group located in position 5 of the aromatic ring which carries it or that RA represents a hydrogen atom or a halogen atom in any position of the aromatic ring;
or RB and RC together form a —CH═ bridge, and the bond which binds it to the aromatic ring is a single bond, provided that Ac represents a CH₂ group and RA represents a hydrogen atom, a hydroxy or alkoxy group located in position of the aromatic ring which carries it;
or RB and RC together form a bond and Ac represents a group

the carbonyl being bonded to the oxygen and the bond joining Ac to the carbon carrying the side chain is a double bond, provided that RA represents a hydrogen atom or a hydroxy or alkoxy group;
when RB represents an alkyl group, X and Y each represent two hydrogen atoms or together with the carbon atom carrying them form a C═O group and RD represents a hydrogen atom or an alkyl group;
when RB and RC form a bridge, X and Y each represent two hydrogen atoms and RD, which only exists when all the bonds of the carbon carrying it are single bonds, represents a hydrogen atom;
   or a pharmaceutically acceptable salt, ester, hydrate, derivative, prodrug or metabolite thereof, for use in the prevention or treatment of an infection by a virus, in particular of the Coronaviridae family, in an individual.

The present invention also relates to a compound of the following formula (V)

wherein
Ar2 and Ar3, which may be the same or different, independently represent a phenyl group or a naphthyl group, or a phenyl group substituted with 1 to 3 groups selected from a hydroxy group, a (C₁-C₆) alkyl, an alkoxy group, a halogen atom and an alkyl substituted with one or more halogen atom;
X ‘and Y’ each represent two hydrogen atoms or together form an oxo group;
RE represents a (C₁-C₆) alkyl group;
   or a pharmaceutically acceptable salt, ester, hydrate, derivative, prodrug or metabolite thereof, for use in the prevention or treatment of an infection by a virus, in particular of the Coronaviridae family, in an individual.

The present invention also relates to a compound of the following formula (VI):

wherein
R3c is a hydrogen atom or a (C₁-C₃) alkyl;
R1c et R2c, which may be the same or different, are selected from H, OH, (C₁-C₃)alkyl, (C₁-C₃) alkoxy, halogen and cyano;
V1 and V2 together form a double bond bonded to an oxygen atom or to a hydroxyimino N—OH radical, or are linked in an ethylenedioxy chain —O—CH₂—CH₂—O—;
Ad represents a valence bond, an oxygen atom, an methylene group or an ethylene group;
m is 0, 1 or 2;
n′ is an integer from 1 to 5;
   or a pharmaceutically acceptable salt, ester, hydrate, derivative, prodrug or metabolite thereof, for use in the prevention or treatment of an infection by a virus, in particular of the Coronaviridae family, in an individual.

The present invention also relates to a compound of the following formula (VII):

wherein
m″ and n″ represent 1 or 2;
Cy represents a (C₃-C₇)cycloalkyl,
Ar4 represent an aryle or a heteroaryl selected from phenyl, naphthyl and thienyl, optionally mono- to tri-substituted by a halogen, a trifluoromethyl, a (C₁-C₃) alkyl, a (C₁-C₃) alkoxy;
   or a pharmaceutically acceptable salt, ester, hydrate, derivative, prodrug or metabolite thereof, for use in the prevention or treatment of an infection by a virus, in particular of the Coronaviridae family, in an individual.

The present invention also relates to a compound of the following formula (VIII):

wherein
One of L and L′ is H and the other is selected from H, F, CI or a nitro group atom, or both L and L′ are CI;
Z represents:

(i) a group of the following structure (1):

wherein
G1 represents a (C₁-C₆) alkyl or a (C₃-C₇) cycloalkyl;
G2 represents a (C₁-C₆) alkyl, a (C₃-C₆)cycloalkyl(C₁-C₃)alkyl, a (C₃-C₇) cycloalkyl, a phenyl, benzyl or phenethyl group optionally substituted on the benzene group of the radical by a halogen atom, or a methoxy or nitro group;
or G1 and G2, together with the nitrogen atom to which they are attached, form a saturated, bridged or spiro mono-nitrogenous heterocycle containing from 5 to 10 carbon atoms; a morpholino group, a piperazino group unsubstituted or substituted in position 4 with a (C₁-C₄) alkyl, with a phenyl, benzyl or phenethyl radical, the benzene group being optionally substituted with a halogen, with a methoxy or nitro group; a group selected from 4-phenyl-1,2,3,6-tetrahydropyridin-1-yl, 4-phenylpiperidino, 4-benzylpiperidino, 4-phenethylpiperidino radicals, the phenyl group of said radicals possibly being unsubstituted or substituted with a halogen, with a methoxy group or a nitro group;

(ii) a group of the following structure (2):

wherein
G3 represents a hydrogen or a hydroxy group;
G4 represents a hydrogen;
or G3 and G4 together constitute one or two bonds so as to form with the carbon atoms to which they are attached a vinylene group or an ethynylene group;
G5 represents a group selected from phenyl, benzyl and phenethyl radicals, the benzene group of said radicals possibly being unsubstituted or substituted with a halogen, a methoxy group or a nitro group;
G6 represents a hydroxy group or a hydrogen atom;
G6 and G7 represent a hydrogen atom or can from a bound;
or G5 and G6 together form an n-pentylene group;

(iii) a group of the following structure (3):

wherein
G3 and G4 are as defined above;
Alk represents a (C₁-C₆)alkyl or a (C₃-C₆)alkenyl;
G8 represents a 1-adamantyl, a (C₃-C₇)cycloalkyl, a (C₃-C₇)cycloalkyl(C₁-C₃)alkyl, or a group selected from the phenyl, benzyl and 2-phenethyl radicals, the benzene group of said radicals may be unsubstituted or substituted with a halogen atom, a methoxy group or a nitro group;
or Alk and G8, which may be the same or different, represent a (C₄-C₆) alkyl group;
G8 is not a (C₃-C₆)cycloalkyl when L is a hydrogen or a fluorine atom or a chlorine atom, L′ is a hydrogen and Alk is a (C₁-C₆) alkyl;

or a pharmaceutically acceptable salt, ester, hydrate, derivative, prodrug or metabolite thereof, for use in the prevention or treatment of an infection by a virus, in particular of the Coronaviridae family, in an individual.

The above compounds can be prepared as described in International publication WO 98/04251, which is incorporated herein by reference, and as described in the publications cited therein, in particular EP376850, EP461986, FR2249659, EP702010, EP707004, EP581677, WO 95/15948.

The present invention also relates to at least one compound of formula (I), (II), (III), (IV), (V), (VI), (VII) or (VIII) as defined above, in particular SR-31747, or a pharmaceutically acceptable salt, ester, hydrate, derivative, prodrug or metabolite thereof for use as defined above, in combination with at least one other compound suitable for the prevention or treatment of an infection by a virus, in particular of the Coronaviridae family, more particularly by SARS-CoV-2.

The present invention also relates to a method for the prevention or treatment of an infection by a virus, in particular of the Coronaviridae family, in an individual, comprising administering to the individual an effective amount of at least one compound of formula (I), (II), (Ill), (IV), (V), (VI), (VII) or (VIII) as defined above, in particular SR-31747, or a pharmaceutically acceptable salt, ester, hydrate, derivative, prodrug or metabolite thereof.

The present invention also relates to a method as defined above, wherein at least one compound of formula (I), (II), (Ill), (IV), (V), (VI), (VII) or (VIII) as defined above, in particular SR-31747, or a pharmaceutically acceptable salt, ester, hydrate, derivative, prodrug or metabolite thereof is administered in combination with at least one other compound suitable for the prevention or treatment of an infection by a virus, in particular of the Coronaviridae family, more particularly by SARS-CoV-2.

The present invention also relates to a pharmaceutical composition, comprising as active substance at least one compound of formula (I), (II), (Ill), (IV), (V), (VI), (VII) or (VIII) as defined above, in particular SR-31747, or a pharmaceutically acceptable salt, ester, hydrate, derivative, prodrug or metabolite thereof for use in the prevention or treatment of an infection by a virus, in particular of the Coronaviridae family, in an individual.

The present invention also relates to a pharmaceutical composition for use as defined above further comprising at least one other compound suitable for the prevention or treatment of an infection by a virus, in particular of the Coronaviridae family, more particularly by SARS-CoV-2.

The present invention also relates to products containing:
at least one compound of formula (I), (II), (Ill), (IV), (V), (VI), (VII) or (VIII) as defined above, in particular SR-31747, or a pharmaceutically acceptable salt, ester, hydrate, derivative, prodrug or metabolite thereof, and
at least one other compound suitable for the prevention or treatment of an infection by a virus, in particular of the Coronaviridae family, more particularly by SARS-CoV-2,

as a combined preparation for simultaneous, separate or sequential use in the prevention or treatment an infection by a virus in an individual.

### DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

### Compounds

The halogen atom according to the invention is preferably selected from the group consisting of F, Cl, Br and I.

Preferably, the cycloalkyl according to the invention is a cyclohexyl.

Preferably, the terms “alkyl” and “alkoxy” refer to linear or branched saturated groups containing from 1 to 6 carbon atoms,

Preferably, the term “substituted” affecting the phenyl and naphthyl substituents means that they can be substituted by 1 to 3 groups preferably selected from a hydroxy group, an alkyl, an alkyl substituted with one or more halogens, an alkoxy group and a halogen atom.

By way of example of pharmaceutically acceptable salt according to the present invention, it is possible to cite salt of mineral or organic acids such as picric acid, oxalic acid mandelic acid or a camphosulfonic acid, as well as hydrochloride salt, hydrobromide salt, succinate salt, sulfate salt, hydrogen sulfate salt, dihydrogen phosphate salt, methanesulfonate salt, methyl sulfate salt, acetate salt, benzoate salt, citrate salt, glutamate salt, maleate salt, fumarate salt, p-toluenesulfonate salt and 2-naphthalenesulfonate salt.

Preferably, the pharmaceutically acceptable salt according to the present invention is the hydrochloride salt.

Preferably, the compounds formulas (I), (II), (Ill), (IV), (V), (VI), (VII) and (VIII) as defined above are selected form the group consisting of:
cis N-cyclohexyl N-ethyl-[3-(3-chloro-4-cyclohexylphenyl)allyl]amine;
trans N-cyclohexyl N-ethyl-[3-(3-chloro-4-cyclohexylphenyl)allyl]amine;
N-cyclohexyl N-ethyl[3-(3-chloro-4-cyclohexylphenyl)propyl]amine;
1-[3-(3-chloro-4-cyclohexylphenyl)allyl]azepane;
trans N, N-dicyclohexyl3-[(3-chloro-4-cyclohexylphenyl)allyl]amine;
N-cyclohexyl N-ethyl[3-(3-chloro-4-cyclohexylphenyl)prop-2-ynyl]amine;
1-(3-chloro-4-cyclohexylphenyI)-3-(cyclohexylethylamino)propan-1-one;
1-(3-chloro-4-cyclohexylphenyI)-3-(cyclohexylethylamino)propan-1-ol;
trans N, N-diethyl-[3-(3-chloro-4-cyclohexylphenyl)allyl]amine;
4-[3-(3-chloro-4-cyclohexylphenyl)propyl]morpholine;
4-[3-chlorohexylphenyl)but-2-enyl]morpholine;
4-[4-(3-chloro-4-cyclohexylphenyl)butyl]morpholine
   or a pharmaceutically acceptable salt, ester, hydrate, derivative, prodrug or metabolite thereof.

Preferably, the compound of formula (I) as defined above is selected from the group consisting of the following compounds:

```
  Cis N-cyclohexyl N-ethyl(chloro-3- cyclohexyl-4 phenyl)-3-propene-2- ylamine also known as SR-31747 or CM 31747
  N,N-dicyclohexyl(chloro-3-cyclohexyl-4- phenyl)-3-propyn-2-ylamine also known as CM 31740
  N-cyclohexyl N-ethyl(chloro-3- cyclohexyl-4-phenyl)-3-propylamine also known as SR 45596 A
  chloro-3-cyclohexyl-4phenyl)-1(N- cyclohexyl N-ethylamino)-3-propanone-1 also known as SR 46232 A
  N-cyclohexyl N-methyl(chloro-3- cyclohexyl-4-phenyl)-3-propyn-2- ylamine also known as CM 31739
  N-cyclohexyl N-methyl(chloro-3- cyclohexyl-4-phenyl)-3-propene-2- ylamine also known as CM 31748
  N-cyclohexyl N-ethyl(chloro-3- cyclohexyl-4 phenyl)-3-propyn-2- ylamine also known CM 31738
  (chloro-3-cyclohexyl-4-phenyl)-1-(N- cyclohexyl N-ethylamino)-3-propanol-1 also known as SR 46233 A
  Chloro-3-(chloro-3-cyclohexyl-4- phenyl)-3-N-cyclohexyl-N-ethyl- propylamine also known as SR 46264 A
```

More preferably, the compound of formula (I) as defined above is SR-31747.

SR-31747 is well known to one of skilled in the art. SR-31747 is also known as N-[(Z)-3-(3-chloro-4-cyclohexylphenyl)prop-2-enyl]-N-ethylcyclohexanamine and can be represented by the following formula (I):

Pharmaceutically acceptable salt, ester, hydrate, derivative, prodrug or metabolite of SR-31747 will be apparent to one of skilled in the art.

An example of a salt of SR-31747 includes the hydrochloride salt.

The term “prodrug” as used herein refers to drug precursors which following administration to the individual, release the drug via chemical and/or physiological process e.g. by hydrolysis and/or enzymatic conversion.

Preferably, in the compound of formula (VIII) as defined above:
L and L′ are as defined above; and
Z represents:

(i) a group of the following structure (1′):

wherein
G1′ represents a (C₁-C₆) alkyl or a (C₃-C₇) cycloalkyl;
G2′ represents a (C₁-C₆) alkyl, a (C₃-C₆)cycloalkyl(C₁-C₃)alkyl, a (C₃-C₇) cycloalkyl, a group selected from a phenyl, benzyl or 2-phenethyl radical, optionally unsubstituted or substituted on the benzene group of the radical with a halogen atom, or a methoxy or nitro group;
or G1′ and G2′, together with the nitrogen atom to which they are attached, form a morpholino group, a pirrolidino, a piperidino, a hexahydroazepino group or a group selected from 4-phenyl-1,2,3,6-tetrahydropyridin-1-yl, 4-phenylpiperidino, 4-benzylpiperidino, 4-phenethylpiperidino radicals, the phenyl group of said radicals possibly being unsubstituted or substituted with a halogen, a methoxy group or a nitro group;

(ii) a group of the following structure (2′):

wherein
G3′ and G4′ are a hydrogen or together form a bound in the trans configuration or preferably cis configuration; G6′ and G7′ are a hydrogen and G5′ is a phenyl or bzneyl, or G5′ and G6′ together form a 1,5-pentylene group;

(iii) a group of the following structure (3′):

wherein G3′ and G4′ are as defined above; Alk″ represents a (C₁-C₆)alkyl,

G8′ represents a 1-adamantyl, a phenyl, benzyl and 2-phenethyl group or

Alk″ and G8′, which may be the same or different, represent a (C₄-C₆) alkyl group.

Preferably, the compound of formula (VIII) as defined above is selected from the group consisting of:
N-benzyl N-methyl-[3-(3-chloro-4-cyclohexyl phenyl) propyl] amine;
1-(3-nitro-4-cyclohexyl phenyl)-3-(4-phenylpiperidino)propanol;
trans 3-[3-(3-nitro-4-cyclohexylphenyl)allyl]-4-phenylpiperidine;
1-[3-(3-chloro-4-cyclohexylphenyl)prop-2-ynyl]-4-phenylpiperidine;
1-(3-(3-chloro-4-cyclohexylphenyl)propyl]-4-phenyl-1,2,3,6-tetrahydro-pyridine;
1-(3-(4-cyclohexylphenyl)propyl]-4-phenylpiperidine;
cis-3-[3-(3-chloro-4-cyclohexylphenyl)allyl]-3-aza-spiro[5.5]undecane;
3-[3-(3-chloro-4-cyclohexylphenyl)propyl]-3-aza-spiro[5.5]undecane;
cis N-adamantan-1-yl-N-ethyl-[3-(3-chloro-4-cyclohexylphenyl)allyl]amine;
4-benzyl-1-[3-(3-chloro-4-cyclohexylphenyl)propyl]piperidine;
1-(3-chloro-4-cyclohexylphenyI)-3-(4-phenylpiperidine-1-yl)propan-1-ol;
cis N-ethyl N-phenyl[3-(3-chloro-4-cyclohexylphenyl)allyl]amine;
N-phenetyl N-methyl-1-3-(3-chloro-4-cyclohexylphenyl)propyl]amine;
N-cyclohexyl N-ethyl-1-[3-(3,5-dichloro-4-cyclohexylphenyl)allyl)amine;
trans N N-dihexyl[3-(3-chloro-4-cyclohexylphenyl)allyl] amine;
   or a pharmaceutically acceptable salt, ester, hydrate, derivative, prodrug or metabolite thereof.

### Virus

The virus according to the invention can be a non-enveloped virus or an enveloped virus. As intended herein, an enveloped virus is a virus that has an outer wrapping or envelope. This envelope comes from the infected cell, or host, in a process called “budding off.” During the budding process, newly formed virus particles become “enveloped” or wrapped in an outer phospholipidic coat that is made from a small piece of the cell's plasma membrane.

Preferably, the virus as defined above is:
a Reoviridae virus, in particular a Rotavirus, more particularly, Human Rotavirus (HRV) or Porcine Rotavirus (PRV),
an Herpesviridae virus, in particular Herpes simplex virus, varicella-zoster virus, cytomegalovirus, Epstein-Barr virus,
a Pleolipoviridae virus, in particular HHPV1, HRPV1, HGPV1, His2V,
a Togaviridae virus, in particular Rubella virus, alphavirus,
an Arenaviridae virus, in particular Lymphocytic choriomeningitis virus,
a Flaviviridae virus, in particular Dengue virus, hepatitis C virus (HCV), yellow fever virus, Zika virus,
an Orthomyxoviridae virus, in particular Influenzavirus A, influenzavirus B, influenzavirus C, isavirus, thogotovirus,
a Paramyxoviridae, in particular Measles virus, mumps virus, respiratory syncytial virus, Rinderpest virus, canine distemper virus,
a Bunyaviridae virus, in particular California encephalitis virus, hantavirus,
a Rhabdoviridae virus, in particular Rabies virus,
a Filoviridae virus, in particular Ebola virus, Marburg virus,
a Coronaviridae, in particular Coronavirus,
a Bornaviridae virus, in particular Borna disease virus,
an Arteriviridae virus, in particular Arterivirus, equine arteritis virus,
a Retroviridae virus, in particular HIV, more particularly HIV-1 or HIV-2,
an Hepadnaviridae virus, in particular hepatitis B virus (HBV).

Preferably, the virus is of the Coronaviridae family.

Preferably, the virus as defined above is of the Alphacoronavirus, Betacoronavirus, Deltacoronavirus, or Gammacoronavirus genus, more preferably of the Betacoronavirus genus, most preferably of the Sarbecovirus or the Merbecovirus sub-genus.

Preferably also the virus as defined above is a human virus, i.e. a virus which can infect a human.

Preferably, the virus as defined above is selected from the group consisting of SARS-CoV, SARS-CoV-2, MERS-CoV and mutants or variants thereof.

Preferably, the virus as defined above is SARS-CoV-2, or a mutant or variant thereof.

SARS-CoV-2 is notably described in Fuk-Woo Chan et al. (2020) Emerging Microbes & Infections 9:221-236, which is incorporated herein by reference, and is also named 2019-nCoV, HCoV-19, SARS2, COVID-19 virus, Wuhan coronavirus, Wuhan seafood market pneumonia virus, and Human coronavirus 2019.

Preferably, the virus as defined above is SARS-CoV-2 and has the genomic sequence defined by NCBI Reference Sequence NC_045512.2 (SEQ ID NO: 1), or the complementary thereof, or is a mutant or variant thereof.

As intended herein, a “mutant or variant” of a virus as defined above, or of a genomic sequence of a virus as defined above, has a genomic sequence or is a nucleotide sequence which has at least 85%, 90%, 95%, 96% 97%, 98%, 99% or 99,5% identity with the genomic sequence of the virus as defined above.

Mutant or variants of SEQ ID NO: 1 can in particular be found on the “NCBI virus” website by searching for SARS-CoV-2 taxid:2697049. A preferred variant of SARS-CoV-2 according to the invention harbours at least one mutation, in particular of the spike protein, selected from the group consisting of K417N, K417T, L452R, T478K, E484K, E484Q, N501Y and D614G. A preferred variant of SARS-CoV-2 according to the invention is a variant of concern, more preferably selected from the group consisting in B.1.1.7, B.1.1.7+E484K, B.1.351, P.1, B.1.617.1, B.1.617.2 and B.1.617.3.

As intended herein, a first nucleotide sequence “having at least X % identity” with a second nucleotide sequence, in particular differs from the second sequence by the insertion, the suppression or the substitution of at least one nucleotide. Besides, the percentage of identity between two nucleotide sequences is defined herein as the number of positions for which the bases are identical when the two sequences are optimally aligned, divided by the total number of bases of the longer of the two sequences. Two sequences are said to be optimally aligned when the percentage of identity is maximal. Besides, as will be clear to one of skill in the art, it may be necessary to add gaps in order to obtain an optimal alignment between the two sequences. In addition, when calculating the percentage of identity between an RNA nucleotide sequence and a DNA nucleotide sequence, an Uracile (U) base and a Thymine (T) base at the same position are considered to be identical.

As intended herein preventing or treating an infection by a virus, in particular of the Coronaviridae family, in an individual, encompasses preventing or treating the symptoms, disorders, syndromes, conditions or diseases, such as pneumonia or COVID-19, associated to the infection by the virus, in particular of the Coronaviridae family, more particularly by SARS-CoV-2.

In particular, the present invention aims at preventing or treating long COVID, which is also known as post-COVID-19 syndrome, post-acute sequelae of COVID-19 (PASC), chronic COVID syndrome (CCS) and long-haul COVID. It is a condition characterized by long-term sequelae—appearing or persisting after the typical convalescence period—of coronavirus disease 2019 (COVID-19).

### Individual

Preferably, the individual is a bird, such as a chicken, or a mammal, such as a human, a canine, in particular a dog, a feline, in particular a cat, an equine, a bovine, a porcine, a caprine, such a sheep or a goat, a mustelidae, such as mink, or a camelidae, more preferably the individual is a human.

Preferably, the individual as defined above is a human aged 50 or more, more preferably 60 or more, even more preferably 70 or more and most preferably 80 or more.

Preferably, the individual as defined above is a male individual.

Preferably, the individual as defined above suffers from at least one other disease or condition, in particular selected from hypertension, diabetes, in particular type 2 diabetes, metabolic syndrome, a cardiovascular disease, in particular ischemic cardiomyopathy, a chronic respiratory disease, or cancer.

Preferably, the individual as defined above is overweight or obese.

According to a usual definition a human individual is considered overweight if its Body Mass Index (BMI, body weight in kg relative to the square of the height in meters) is higher than or equal to 25 kg/m² and less than 30 kg/m² and the individual will be said obese if his BMI is higher than or equal to 30 kg/m². The individual according to the invention may notably present with severe obesity, in particular characterized in human by a BMI higher than or equal to 35 kg/m².

More generally, it is preferred that the individual as defined above is a human and has a BMI higher than or equal to 25 kg/m², 26 kg/m², 27 kg/m², 28 kg/m², 29 kg/m², 30 kg/m², 31 kg/m², 32 kg/m², 33 kg/m², 34 kg/m², 35 kg/m² or 40 kg/m².

Besides, the individual as defined above may also have an abdominal obesity, corresponding in particular to a visceral adipose tissue excess. According to a usual definition a male human individual has an abdominal obesity if the abdominal perimeter is higher than or equal to 94 cm, in particular higher than 102 cm and a female human individual has an abdominal obesity if the abdominal perimeter is higher than or equal to 80 cm, in particular higher than 88 cm. The abdominal perimeter measure is well known to one of skilled in the art: abdomen circumference is thus preferably measured midway between the last floating rib and the top of the iliac crest in a standing individual in gentle expiration.

It is particularly preferred that the individual as defined above is a man and presents with an abdominal perimeter higher than or equal to 90 cm, 91 cm, 92 cm, 93 cm, 94 cm, 95 cm, 96 cm, 97 cm, 98 cm, 99 cm, 100 cm, 101 cm or 102 cm. It is also preferred that the individual according to the invention is a woman and presents with an abdominal perimeter higher than or equal to 75 cm, 76 cm, 77 cm, 78 cm, 79 cm, 80 cm, 81 cm, 82 cm, 83 cm, 84 cm, 85 cm, 86 cm, 87 cm or 88 cm.

Preferably, the individual according to the invention is afflicted with COVID-19 or is at risk of being afflicted with COVID-19.

### Additional Compound

Preferably, the other compound suitable for the prevention or treatment of an infection by a virus, in particular of the Coronaviridae family, more particularly by SARS-CoV-2, is selected from the group consisting of chloroquine, hydroxychloroquine, azithromycin, remdesivir, ribavirin, penciclovir, favipravir, a cysteine protease inhibitor, in particular a cathepsin L inhibitor, such as camostat and nafamostat, nitazoxanide, thalidomide, fingolimod, carrimycin, lopinavir/ritonavir, methylprednisolone, dexamethasone, bevacizumab, tocilizumab, sarilumab, N-acetylcysteine, recombinant human interferon a1β, arbidol, eculizumab, darunavir, cobicistat, meplazumab, danoprevir, peginterferon alfa-2a, oseltamivir, nicotine, chlorpromazine, intravenous immunoglobulins, a statin, an angiotensin-converting enzyme inhibitor (ACEI)/angiotensin II receptor blocker (ARB), such as losartan, a calcium channel blocker (CCB), such as amlodipine besylate, an amino-bisphosphonate, such as zoledronic acid, ivermectin, colchicine, clofoctol, GS-441524, MK-711, molnupiravir and pharmaceutically acceptable salts, esters, hydrates, derivatives, prodrugs or metabolites thereof.

Preferably, the other compound suitable for the prevention or treatment of an infection by a virus of the Coronaviridae family, in particular by SARS-CoV-2, is a statin selected from the group consisting of atorvastatin, cerivastatin, fluvastatin, lovastatin, mevastatin, pitavastatin, pravastatin, rosuvastatin, and simvastatin, more preferably it is simvastatin.

### Pharmaceutical Composition

The compounds of formulas (I), (II), (Ill), (IV), (V), (VI), (VII), and (VIII), in particular SR-31747, or a pharmaceutically acceptable salt, ester, hydrate, derivative, prodrug or metabolite thereof, optionally combined with at least one other compound suitable for the prevention or treatment of an infection by a virus, in particular of the Coronaviridae family, more particularly by SARS-CoV-2, can be comprised in a pharmaceutical composition which can comprise at least one pharmaceutically acceptable vehicle or excipient. The pharmaceutically acceptable vehicle or excipient can be selected from dispersants, solubilizers, stabilizers, preservatives, etc. Besides, pharmaceutically acceptable vehicle or excipient which can be used in formulations, in particular liquid and/or injectable formulations, are preferably selected from sucrose, lactose, starch, methylcellulose, hydroxymethylcellulose, carboxymethylcellulose, croscarmellose sodium, lactose monohydrate, magnesium stearate, microcrystalline cellulose, povidone, sodium lauryl sulfate, mannitol, gelatin, lactose, vegetable oils, acacia gum, liposomes, etc.

### Administration

As intended herein “combined” or “in combination” means that the composition as defined above, is administered at the same time than the additional compound as defined above, either together, i.e. at the same administration site, or separately, or at different times, provided that the time period during which the composition as defined above exerts its pharmacological effects on the individual and the time period during which the additional compound exerts its pharmacological effects on the individual, at least partially intersect.

The compounds of formulas (I), (II), (Ill), (IV), (V), (VI), (VII), and (VIII), in particular SR-31747, or a pharmaceutically acceptable salt, ester, hydrate, derivative, prodrug or metabolite thereof or the pharmaceutical composition as defined above can be administered orally, parenterally, mucosally or cutaneously. The parenteral route preferably comprises subcutaneous, intravenous, intramuscular or intraperitoneal administration, although the latter is rather reserved for animals. The mucosal route preferably comprises buccal administration, sublingual administration, nasal administration, pulmonary administration or administration via the rectal mucosa. The cutaneous route advantageously comprises the dermal route, in particular via a transdermal device, typically a patch.

The compounds of formulas (I), (II), (Ill), (IV), (V), (VI), (VII), and (VIII), in particular SR-31747, or a pharmaceutically acceptable salt, ester, hydrate, derivative, prodrug or metabolite thereof or the pharmaceutical composition as defined above can be formulated in the form of injectable suspensions, gels, oils, tablets, suppositories, powders, gel capsules, capsules, aerosols, etc., optionally by means of galenical forms or of devices which provide sustained and/or delayed release. For this type of formulation, an agent such as cellulose, carbonates or starches is advantageously used.

The compounds of formulas (I), (II), (Ill), (IV), (V), (VI), (VII), and (VIII), in particular SR-31747, or a pharmaceutically acceptable salt, ester, hydrate, derivative, prodrug or metabolite thereof or metabolite thereof or the pharmaceutical composition as defined above can be administered to the individual as defined above at a dose between 1 mg and 1 g, preferably between 5 mg and 500 mg, even more preferably between 50 mg and 125 mg, and most preferably 75 mg, of SR-31747 or a pharmaceutically acceptable salt, ester, hydrate, derivative, prodrug or metabolite thereof as defined above. Of course, those skilled in the art are able to adjust the dose of SR-31747 or a pharmaceutically acceptable salt, ester, hydrate, derivative, prodrug or metabolite thereof as defined above according to the weight of the individual to be treated. Preferably, the dosage range of The compounds of formulas (I), (II), (Ill), (IV), (V), (VI), (VII), and (VIII), in particular SR-31747, or a pharmaceutically acceptable salt, ester, hydrate, derivative, prodrug or metabolite thereof is from 1 mg and 100 mg/kg/day, preferably between 5 mg and 500 mg/kg/day, even more preferably between 5 and 30 mg/kg/day, and most preferably 25 mg/kg/day.

The invention will be further explained by the following non-limiting figure and Examples.

### DESCRIPTION OF THE FIGURES

FIG. 1 represents the effect of SR-31747 on the replication cycle of SARS-Cov-2 with the % of infection inhibition of SR-31747 on the y-axis and the log[SR-31747] on the x-axis.

FIG. 2 represents the mean cumulative clinical score (vertical axis) of hamsters infected by SARS-CoV-2 and treated by SR-31747 (empty squares), infected by SARS-CoV-2 and treated by diluent (negative control) (circles), non-infected and treated by SR-31747 (triangles) and non-infected and treated by diluent (full squares), on days 0, 1, 2, 3 and 4 post-infection.

FIG. 3 and FIG. 4 represent the body temperature (in ° C., vertical axis) of hamsters infected by SARS-CoV-2 and treated by SR-31747 (group 1, empty squares), infected by SARS-CoV-2 and treated by diluent (negative control) (group 2, circles), non-infected and treated by SR-31747 (group 3, triangles) and non-infected and treated by diluent (group 4, full squares), respectively on days 2 and 4 post-infection.

FIG. 5 represents the percentage of hamsters that did not find hidden food (%, vertical axis) as a function of the latency time to find the hidden cereals (in seconds, horizontal axis) for hamsters infected by SARS-CoV-2 and treated by SR-31747 (empty squares), infected by SARS-CoV-2 and treated by diluent (negative control) (circles), non-infected and treated by SR-31747 (triangles) and non-infected and treated by diluent (full squares).

FIG. 6 represents the lung weight (in g, vertical axis) at 4 days post-infection of hamsters infected by SARS-CoV-2 and treated by SR-31747 (group 1, empty squares), infected by SARS-CoV-2 and treated by diluent (negative control) (group 2, circles), non-infected and treated by SR-31747 (group 3, triangles) and non-infected and treated by diluent (group 4, full squares).

### EXAMPLES

### Example 1

The efficacy of SR-31747 in inhibiting Coronaviridae virus infections can be determined as follows.

1. Efficacy of SR-31747 on replication of live virus

SR-31747 is first tested for its capacity to inhibit the replication of different luciferase-encoding coronaviruses:

| Virus | Target cell lines |
|---|---|
| MHV*-Luciferase | murine cells (LR7) |
| FIPV**-Luciferase | feline cells (FCWF) |
| PEDV**-Luciferase | african green monkey cells (Vero) |

*MHV is a prototype coronavirus of the Betacoronavirus genus
**FIPV and PEDV are both members of the Alphacoronavirus genus

1.1. SR-31747 toxicity to Cells

In a first step the toxicity of SR-31747 on the three target cells is tested at different concentrations using a standard WST assay, in which the tetrazolium salt WST-1 is cleaved to formazan by the succinate-tetrazolium reductase system (which belongs to the respiratory chain of the mitochondria) only by metabolically intact cells. Formazan concentration can be determined by absorbance measurements which correlate directly to the number of viable cells.

1.2. Effect of pre-treatment with SR-31747 on virus replication

In a second step, the effect of SR-31747 on viral replication is tested by either pre-treating viruses or target cells by SR-31747:
Virus pre-treatment: Target cells are infected (MOI: 0.01) with viruses pre-treated for 1 hour with 3 SR-31747 concentrations, and luciferase activity (proxy for virus infection) in cell lysates is measured at different times post infection (T=0, 3, 6, 9, 24, 32 and 48 hours post infection); virus titers in supernatants (collected at 10 hours post infection) are assessed by TCID50 (50% tissue culture infective dose) analysis.
Cell pre-treatment: Target cells are pre-treated with 3 SR-31747 concentrations for 1 hour, and luciferase activity in cell lysates is measured at different times post infection (e.g. T=0, 3, 6, 9, 24, 32 and 48 hours post infection); virus titers in supernatants (collected at indicated time-points) are assessed by TCID50 analysis.

1.3. Effect of treatment with SR-31747 on virus replication In a third step, the effect of SR-31747 is tested by treating infected target cells:
Target cells are infected with the three target viruses (high MOI=2) and SR-31747 is added to target cells at −2 h prior to or 2, 4, 6, 8 h after infection. Luciferase activity is measured at 10 hours post infection, and virus titers in supernatants (collected at 10 hours post infection) are assessed by TCID50 analysis.

2. Effect of SR-31747 on the entry spike-pseudotyped virus in target cells

The capacity of SR-31747 to inhibit the entry of luciferase-encoding vesicular stomatitis virus (VSV) pseudotyped by the spike protein of different Coronaviridae viruses in target cells is tested in a VSV pseudotyped particle (VSVpp) entry assay.

| VSVpp | Target cell lines |
|---|---|
| SARS-CoV spike VSVpp | Vero |
| SARS-CoV-2 spike VSVpp | Vero |
| HCoV-OC43 spike VSVpp | HRT-18 |
| MERS-CoV spike VSVpp | Vero |
| VSV-G control | Vero/HRT-18 |

Briefly, target cell lines are pre-treated with 3 SR-31747 concentrations for 1 hour, and luciferase activity (proxy for virus infection) in cell lysates is measured at T=24 hours post infection.

### Example 2

The effect of SR-31747 on the replication cycle of SARS-Cov-2 has been determined as follows.

1. Protocol
Cell lines: lung cancer A549 stably transfected with a lentiviral construct bearing the human ACE2 receptor cultured in DMEM with 10% serum and 1% penicillin/streptomycin.
Format: 384 well plate
MOI (multiplicity of infection)=0,1, SARS-CoV-2
Total incubation time: 72 hours
Positive control compound for viral replication used: remdesivir 10 μM
Positive control compound for cytotoxicity used: camtothecine 10 μM
Negative control: DMSO 0,5%

Five concentration of the SR-31747 in triplicate were used: 30 μM, 10 μM, 3 μM, 1 μM, and 0.3 μM.

Cells (50% confluency) were first preincubated with SR-31747 2h before infection with the virus for 1 h.

The inoculum was then removed and 40 μL of medium with the drugs were added on the cells.

After 72h of incubation, the supernatant was recovered and the measurement of viral replication was carried out by quantitative RT-PCR in the presence and absence of drugs.

Detection: supernatant PCR-N gene region: 5′-TAATCAGACAAGGAACTGATTA-3 (forward) (SEQ ID NO: 2) and 5′-CGAAGGTGTGACTTCCATG-3′ (reverse) (SEQ ID NO: 3); Luna Universal One-step RT- qPCR kit (NEB) in an applied biosystems QuantStudio thermocycler. The quantity of viral genomes is expressed as PFU (plaque forming unit) equivalents, and was calculated by performing a standard curve with RNA derived from a viral stock with a known viral titer (plaque forming unit).

In parallel, cell viability was assessed after 72h incubation with SR-31747 using the CellTiter Glo kit from Promega which measures the cellular ATP concentration of live cells.

Raw data are normalized against appropriate negative (0%) ad positive (100%) controls and are expressed in % of viral replication inhibition or % cytotoxicity.

The curve fit is performed using the variable Hill Slope model or the four-parameter logistic curve:

$$Response = Baseline response + \frac{( Maximum response - Baseline response )}{1 + {10}^{( LogBcso - logConcentration ) \cdot HillSlope}}$$

where.
Response is the measured response on the Y axis;
Baseline response is the maximum response at the bottom of the plateau;
Maximum response is the maximum response at the top of the plateau;
Ic50 is the concentration at 50% response;
Concentration is the measures drug concentration on the X axis;
Hill Slope is the Hill coefficient that describes the steepness of the curve.

2. Results

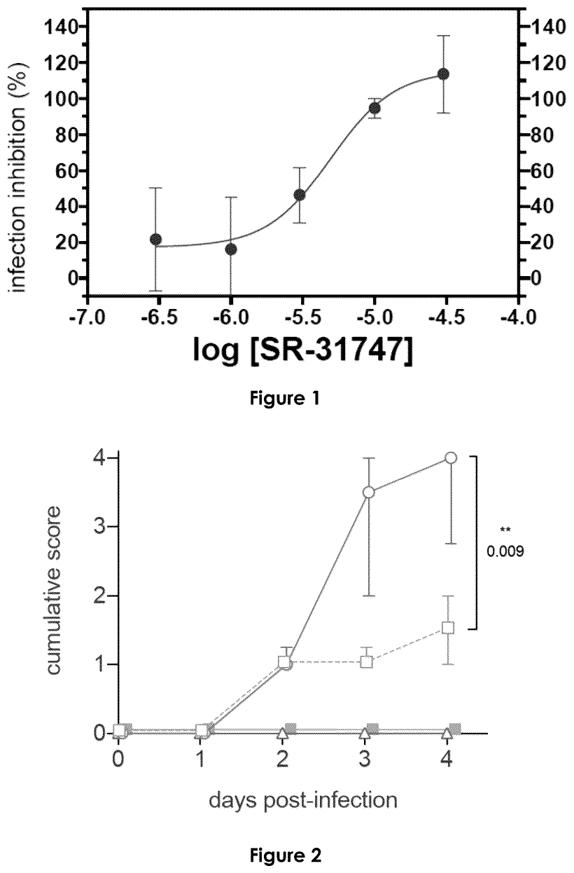

The results are summarized in FIG. 1 and in the table below:

| Molecule name | RTqPCR: IC50 (μM) | Cytotoxicity: IC50 (μM) |
|---|---|---|
| SR-31474 | 2.88 | 11 |

### Example 3

The EC50 and CC50 of SR-31747 were determined in an in vitro model of infection by human α-coronavirus 229E.

1. Method

Briefly, a dilution series of SR-31747 (8-point, half-log dose titration, 30 μM-10 nM) was added to 16HBE cells. Vehicle and positive control (remdesivir, 8-point, half-log dose titration, 20 μM-6.4 nM) wells were set up to control for any influence of the compounds alone on cell viability. Cells were visually inspected for the appearance of any cytopathic effects (CPE). A cell viability assay was performed once CPE was complete.

The readouts were:
EC50: The concentration which results in 50% viral inhibition, following the addition of compounds;
CC50: The concentration which results in 50% cell viability, following the addition of compounds;
Selectivity index: Calculated as CC50/EC50.

Protocol:
1. 16HBE cells permissible to infection by α-coronavirus 229E were grown and seeded into 96-well plates to a confluency of 80-90%.
2. The compounds SR-31747 and remdesivir (positive control) were serially diluted half-log into 8 concentrations in total and added to cells for 1 hour at 37° C.
3. After 1 hour, α-coronavirus 229E was added at 100xTCID50. A mock infection of blank media was added to the uninfected controls.
4. After infection, the virus/compound was removed and an overlay medium was added, with equivalent concentrations of the compounds.
5. Cells were incubated until extensive cytopathic effects (CPE) were seen in the infected control wells (about 5 days).
6. Once CPE was observed, supernatants were taken and stored at −80° C.
7. A cell viability assay was then performed on all conditions, with cells receiving the treatment compared to the vehicle treated and uninfected controls. Calculations are as follows:

% viral inhibition=[(A-B)/(C-B)] x100, where:
A: mean optical density of test, B: mean optical density of virus controls, C: mean optical density of cell controls.
Negative values occur when A<B, due either to natural variation or compound toxicity.

% cell viability=X/Y x100, where:
X: mean optical density of test; Y: mean optical density of cell controls.

2. Results

| | |
|---|---|
| SR-31747 (μM) | Remdesivir (μM) |

| Virus | EC50 | CC50 | EC50 | CC50 |
|---|---|---|---|---|
| a-coronavirus | 1.362 | 6.95 | 0.34 | >20 |
| (229E) | | | | |

SR-31747 shows activity against α-coronavirus, strain 229E, with an EC50 of 1.362 μM. The selectivity index is calculated to be 5.

### Example 4

The effect of SR-31747 in SARS-CoV-2-infected golden hamsters was investigated.

1. Material and method

SR-31747 was used freshly diluted in a diluent consisting of 95% water, 5% ethanol and 5% tween-80.

24 male hamsters RjHan:AURA SPF were acclimated during 7 days and distributed in 4 groups of 6 hamsters each:
Group 1: Infected by SARS-CoV-2 and treated by SR-31747
Group 2: Infected by SARS-CoV-2 and treated by diluent (negative control)
Group 3: Non-infected and treated by SR-31747
Group 4: Non-infected and treated by diluent

On day 0, hamsters were infected intranasally by SARS-CoV-2 and received a first intraperitoneal injection of SR-31747 at 40 mg/kg.

On days 1, 2 and 3 the hamsters received an intraperitoneal injection of SR-31747 at 40 mg/kg. On day 4 sera and lungs from the hamsters were sampled.

From day 0 of day 4, the body temperature of the hamsters was measured at both flanks using a touchless infrared thermometer and a cumulative clinical score was determined (ruffled fur (no=0 or yes=1), slow movements (no=0, yes=1), apathy (no=0, yes=1), absence of rearing/exploration (no=0, yes=1)).

An olfactory test was conducted on day 3 preceded by a day of fasting. Briefly, cereals were buried in the litter of an individual cage and the latency to find the hidden cereals by the hamsters was measured.

2. Results
Cumulative clinical score

The evolution of the clinical scores from day 0 to day 4 is shown in FIG. 2.

SR-31747 significantly reduces the cumulative clinical score in hamsters infected by SARS-CoV-2 on day 4 (p=0,009, Mann-Whitney test).
Body temperature

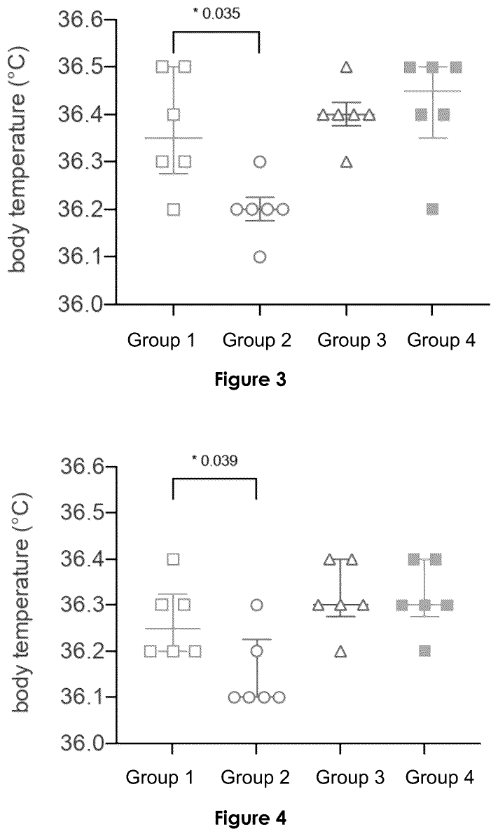

The body temperature of the animals on day 2 and on day 4 is shown in FIGS. 3 and 4 respectively.

While the body temperature is decreased in animals infected by SARS-CoV-2 decreases, treatment by SR-31747 significantly increases the temperature of the hamsters.
Olfactory test

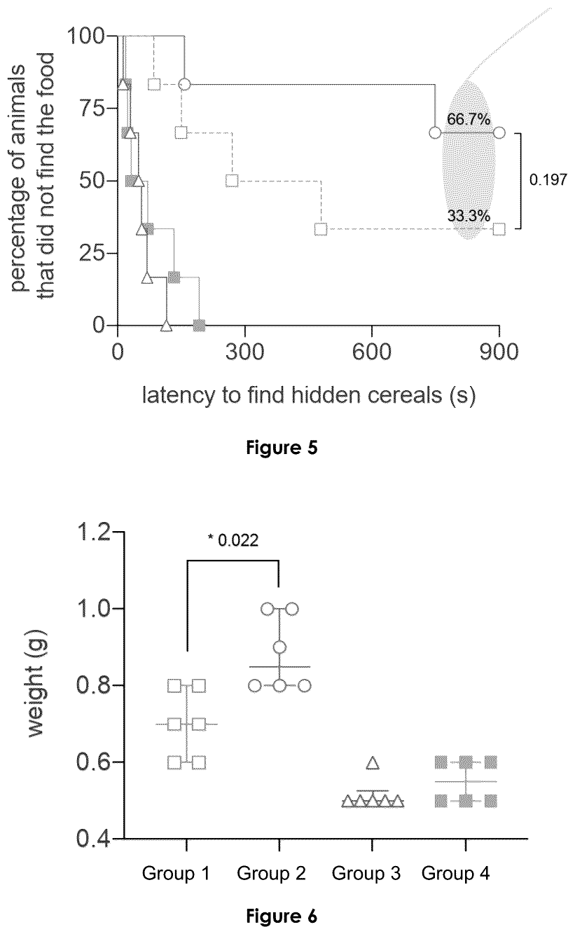

The latency to find hidden cereals, which is representative of an olfactory dysfunction, is shown in FIG. 5.

Treatment with SR-31747 alleviates the olfactory dysfunction induced by SARS-CoV-2.
Lung weight on day 4

The weight of the sampled lungs at the end of the experiment is shown in FIG. 6.

Infection by SARS-CoV-2 is associated to an increase in lung weight which is significantly alleviated by treatment with SR-31747.

In view of the foregoing, SR-31747 treats the symptoms of SARS-CoV-2 infection in an hamster model of COVID-19.

1. A method for preventing or treating an infection by a virus in an individual, comprising administering a compound of formula (I)
wherein
R1 is a hydrogen atom or a halogen atom;
R2 is a cyclohexyl or a phenyl;
R3 is a cycloalkyl having 3 to 6 carbon atoms;
R4 is a hydrogen atom, an alkyl having 1 to 6 carbon atom or a cycloalkyl having 3 to 6 carbon atoms; and
A is —CO—CH₂—, —CH(C₁)—CH₂—, —CH(OH)—CH₂—, —CH₂—CH₂—, —CH═CH—, or —C═C—
or a pharmaceutically acceptable salt, ester, hydrate, derivative, prodrug or metabolite thereof to the individual.

2. The method of claim 1, wherein the compound of formula (I) is

3. The method of claim 1, wherein the compound of formula (I) is SR-31747.

4. The method of claim 1, wherein the virus is of the Coronaviridae family.

5. The method of claim 1, wherein the virus is SARS-CoV, SARS-CoV-2, MERS-CoV or mutants or variants thereof.

6. The method of claim 1, wherein the virus is SARS-CoV-2, or a mutant or variant thereof.

7. The method of claim 1, wherein the individual is aged 50 or more.

8. The method of claim 1, wherein the individual suffers from at least one other disease or condition.

9. The method of claim 1, further comprising administering at least one other compound suitable for the prevention or treatment of an infection by a virus.

10. A method for preventing or treating an infection by a virus in an individual, comprising administering a pharmaceutical composition comprising a compound of formula (I) or a pharmaceutically acceptable salt, hydrate, or prodrug thereof as active substance, to the individual.

11. The method of claim 10, wherein the virus is of the Coronaviridae family.

12. The method of claim 10, wherein the virus is selected from the group consisting of SARS-CoV, SARS-CoV-2, MERS-CoV or mutants or variants thereof.

13. The method of claim 10, wherein the virus is SARS-CoV-2, or a mutant or variant thereof.

14. The method of claim 10, further comprising administering at least one other compound suitable for the prevention or treatment of an infection by a virus.

15. A process for preventing or treating an infection by a virus in an individual comprising administering simultaneously, separately, or sequentially:
a compound of formula (I) or a pharmaceutically acceptable salt, ester, hydrate, derivative, prodrug or metabolite thereof, and
at least one other compound suitable for the prevention or treatment of an infection by a virus, to the individual.

16. The method of claim 1, wherein the virus is of the Betacoronavirus genus.

17. The method of claim 8, wherein the at least one other disease or condition is hypertension, diabetes, a cardiovascular disease, a chronic respiratory disease, and/or cancer.

18. The method of claim 10, wherein the virus is of the Betacoronavirus genus.

19. The method of claim 10, wherein the individual suffers from at least one other disease or condition.

20. The method of claim 19, wherein the at least one other disease or condition is hypertension, diabetes, a cardiovascular disease, a chronic respiratory disease, and/or cancer.
